(19) United States
(12) Patent Application Publication
(10) Pub. No.: US 2024/0192140 A1
(43) Pub. Date: Jun. 13, 2024

(54) BIO-ENHANCED REMEDIATION AND OIL RECOVERY MODEL SYSTEM AND METHODS

(71) Applicant: REGENTS OF THE UNIVERSITY OF MINNESOTA, Minneapolis, MN (US)

(72) Inventors: Peter K. Kang, Minneapolis, MN (US); Sang Hyun Lee, Minneapolis, MN (US); Cara M. Santelli, St. Paul, MN (US); Shreya Srivastava, Minneapolis, MN (US)

(21) Appl. No.: 18/536,537

(22) Filed: Dec. 12, 2023

Related U.S. Application Data
(60) Provisional application No. 63/431,890, filed on Dec. 12, 2022.

(51) Int. Cl. G01N 21/88; C02F 3/34; C12M 1/00; C12M 3/06; C12N 1/14; G01N 21/64

(52) U.S. Cl. CPC G01N 21/8803; C02F 3/347; C12M 23/16; C12M 29/14; C12N 1/14; G01N 21/64; C02F 2101/32

(57) ABSTRACT

A bio-enhanced remediation model system generally includes a microfluidic device, a fluid source in fluid communication with the microfluidic device, and an imager in visual communication with a portion of the microfluidic device. Generally, the microfluidic device includes a main flow channel having an inlet, an outlet, and a main flow channel width; one or more microfluidic beds in fluid communication with the main flow channel, at least one microfluidic bed having a plurality of structures that define secondary channels, at least a portion of the secondary channels having a width narrower than the main flow channel width; an aqueous phase dispersed within at least a portion of the main flow channel; and a non-aqueous phase liquid (NAPL) dispersed within at least a portion of the secondary channels.

### CROSS-REFERENCE TO RELATED APPLICATION

This application claims the benefit of U.S. Provisional Patent Application No. 63/431,890, filed Dec. 12, 2022, which is incorporated herein by reference in its entirety.

### SUMMARY

This disclosure describes, in one aspect, a bio-enhanced remediation model system. Generally, the bio-enhanced remediation model system comprises a microfluidic device, a fluid source in fluid communication with the microfluidic device, and an imager in visual communication with a portion of the microfluidic device. Generally, the microfluidic device includes a main flow channel having an inlet, an outlet, and a main flow channel width; one or more microfluidic beds in fluid communication with the main flow channel, at least one microfluidic bed having a plurality of structures that define secondary channels, at least a portion of the secondary channels having a width narrower than the main flow channel width; an aqueous phase dispersed within at least a portion of the main flow channel; and a non-aqueous phase liquid (NAPL) dispersed within at least a portion of the secondary channels.

In one or more embodiments, the bio-enhanced remediation model system further includes a computer in electronic communication with the imager.

In one or more embodiments, the microfluidic device includes two or more microfluidic beds in fluid communication with the main flow channel.

In one or more embodiments, the main flow channel has a width of from 1 μm to 10 cm.

In one or more embodiments, the bio-enhanced remediation model system further includes a filamentous microbe. In one or more of these embodiments, the filamentous microbe is a hyphal fungus. In one or more of these embodiments, the filamentous microbe is genetically modified.

In one or more embodiments, the bio-enhanced remediation model system includes two or more species of microbes, at least one of which is a filamentous microbe.

In another aspect, this disclosure describes a method for identifying a filamentous microbe as a bio-enhanced remediation microbe or a bio-enhanced oil recovery microbe. Generally, the method includes providing a bio-enhanced remediation model system, at least one filamentous microbe into the aqueous phase of the bio-enhanced remediation model system, incubating the bio-enhanced remediation model system under conditions effective to allow the filamentous microbe to colonize the main flow channel, observing hyphae in the secondary channels of at least one microfluidic bed, and identifying the filamentous microbe as a bio-enhanced remediation microbe. Alternatively, the method includes providing a bio-enhanced remediation model system, at least one filamentous microbe into the aqueous phase of the bio-enhanced remediation model system, incubating the bio-enhanced remediation model system under conditions effective to allow the filamentous microbe to colonize the main flow channel, observing hyphae in the secondary channels of at least one microfluidic bed, and identifying the filamentous microbe as a bio-enhanced oil recovery microbe.

In one or more embodiments, the non-aqueous phase liquid (NAPL) provided in the bio-enhanced remediation model system includes a compound present at a remediation site of interest.

In one or more embodiments, the method may be performed using a microbial composition that includes a plurality of microbes, at least one of which is a filamentous microbe.

In another aspect, this disclosure describes a method of comparing bio-enhanced remediation of a non-aqueous phase liquid (NAPL) by different bio-enhanced remediation microbial compositions. Generally, the method includes providing a first bio-enhanced remediation model system that includes the NAPL in the microfluidic bed, introducing a first microbial composition into the aqueous phase of the first bio-enhanced remediation model system, the first microbial composition including at least one filamentous microbe, incubating the first bio-enhanced remediation model system under conditions effective to allow members of first microbial composition to colonize the main flow channel of the first bio-enhanced remediation model system, measuring hyphae infiltration into the NAPL of at least one microfluidic bed of the first bio-enhanced remediation model system, repeating the previous steps using a second bio-enhanced remediation model system and a second microbial composition, and selecting for bio-enhanced remediation of the NAPL the microbial composition with greater hyphae infiltration into the NAPL.

In one or more embodiments, the first microbial composition and/or the second microbial composition comprises a plurality of different microbes, at least one of which is a filamentous microbe.

In another aspect, this disclosure describes a method of comparing bio-enhanced remediation of a non-aqueous phase liquid (NAPL) by different bio-enhanced remediation microbial compositions. Generally, the method includes providing a first bio-enhanced remediation model system that includes the NAPL in the microfluidic bed, introducing a first microbial composition into the aqueous phase of the first bio-enhanced remediation model system, the first microbial composition including at least one filamentous microbe, incubating the first bio-enhanced remediation model system under conditions effective to allow members of first microbial composition to colonize the main flow channel of the first bio-enhanced remediation model system, measuring removal of NAPL from at least one microfluidic bed of the first bio-enhanced remediation model system, repeating the previous steps using a second bio-enhanced remediation model system and a second microbial composition, and selecting for bio-enhanced remediation of the NAPL the microbial composition with greater removal of the NAPL.

In one or more embodiments, the first microbial composition and/or the second microbial composition comprises a plurality of different microbes, at least one of which is a filamentous microbe.

In another aspect, this disclosure describes a method of bio-enhanced remediation. Generally, the method includes introducing into a bio-enhanced remediation site a bio-enhanced remediation composition that includes a filamentous microbe.

In one or more embodiments, the filamentous microbe is genetically modified to metabolize at least one contaminant of the bio-enhanced remediation site.

In one or more embodiments, the composition comprises a plurality of different microbes, at least one of which is a filamentous microbe. In one or more of these embodiments, at least one microbe is genetically modified.

In another aspect, this disclosure describes a bio-enhanced remediation composition that includes a filamentous microbe.

In one or more embodiments, the filamentous microbe is genetically modified.

In one or more embodiments, the composition further includes a second species of microbe. In one or more of these embodiments, the second species of microbe is a filamentous microbe.

In one or more embodiments, at least one species of microbe is genetically modified. In one or more of these embodiments, the genetic modification increases consumption of a non-aqueous phase liquid (NAPL), decreases the ability of the microbe to metabolize carbon sources other than the NAPL, increases the microbe's fitness under selected environmental conditions, integrates a pollution receptor into a fluorescent protein secretion circuit, or integrates a pollution receptor into a bioluminescence circuit.

In another aspect, this disclosure describes a method of recovering oil from an oil recovery site. Generally, the method includes introducing into the oil recovery site a bio-enhanced oil recovery composition that includes a filamentous microbe.

In one or more embodiments, the composition includes a plurality of different microbes, at least one of which is a filamentous microbe.

In another aspect, this disclosure describes a bio-enhanced oil recovery composition that includes a filamentous microbe.

In one or more embodiments, the composition further includes a second species of microbe. In one or more of these embodiments, the second species of microbe is a filamentous microbe.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1. Exemplary hydrocarbon-degrading fungus and microfluidics system. (A) Microscopic image of the branching fungus (Penicillium sp.). (B) Schematic of an exemplary microfluidics system employed in this study. (C) Detailed structure and dimension of the exemplary “Fracture-on-a-chip” system used in this study.

FIG. 2. Snapshots of fracture-on-a-chip after 65 hour-injection. (A) fungal suspension; (B) non-branching bacterial suspension; and (C) sterile media at a constant flow rate of 0.7 L/min. Brown shade represents oil saturated area.

FIG. 3. Snapshots of “fracture-on-a-chip” at pressure difference (ΔP) near capillary pressure (40 mbar). (A) At 27 hours; T=27 hr. (B) At 27.5 hours; T=27.5 hr. Fungal colonization of the main flow channel causes pressure build-up upstream of the choking point (red circle), which induces invasion of the water phase, noted by red arrows, into low porosity regions.

FIG. 4. “Fracture-on-a-chip” at pressure difference (ΔP) near capillary pressure (40 mbar). (A) Plot of ΔP over time. The dashed line indicates the estimated capillary pressure. (B) Close-up image of the clogged region (right red circle in FIGS. 3A & 3B) showing differential invasion with respect to the degree of clogging.

FIG. 5. Oil removal is accelerated when fungal hyphae directly interact with the oil. Oil-water interfaces that are penetrated by fungal hyphae are selectively receded. (A) Blue lines indicate fungal hyphae at initial monitoring, T=0 hr. (B) Blue lines indicate fungal hyphae at initial monitoring, T=0 hours. Red lines indicate fungal hyphae after 24 hours of monitoring, T=24 hours.

FIG. 6. The fungal strain isolated in this study is not only capable of degrading naphthalene but also can use vegetable oil as a carbon source. Inoculation of fungi in M10 and vegetable oil mixture resulted in the growth of fungi, indicating direct consumption of the model oil by the fungi, which could contribute to oil removal from the chip.

FIG. 7. Hyphae are hydrophilic. As fungal hypha approaches and pokes into an oil blob, the shape of the interface moves away from the hypha. The contact angle, 0, of oil on the hypha surface in the water phase seems larger than 900 indicating that oil is non-wetting to hyphae and hypha is water-wet or hydrophilic. Such property can facilitate the mechanical displacement of oil from the chip. (A) At initial monitoring, T=0 hr. (B) After 1.5 hours of monitoring, T=1.5 hr.

FIG. 8. Proposed mechanism of fungal bio-enhanced remediation. (A) First, injected fungi can colonize the main flow channel (e.g., fracture) and build up pressures upstream above the capillary pressure causing invasion of water phase into pores and subsequent displacement of oil out of the pore. (B) Second, fungal colonies grow and extend their hyphae into the oil phase facilitating direct consumption and hydrophilic hyphae-enhanced oil displacement. Note that in the presence of a strong pressure gradient in the water phase (i.e., high flow rate), the former effect may dominate.

FIG. 9. Fungi-enhanced oil removal in randomized porous media chip. (A) Blueprint for the randomized chip. (B) Oil removal by fungal colonization. Blue color represents regions invaded by water and the location of the fungal colony along the channel is indicated by the red arrow in the inset. (C) Plots of pressure drop, and volume % of oil removed from the chip with respect to time.

FIG. 10. Oil removal is accelerated when fungal hyphae directly interact with the oil. Similar to FIG. 5, but penetration of multiple filaments per throat induced oil displacement and water invasion in porous media structure with larger pore throat size (100 μm).

FIG. 11. Hydrophilicity of fungi is confirmed via Microbial Adsorption To Hydrocarbon (MATH) test and visual observation of contact angle. (A) Illustration of MATH methodology. (B) Result of MATH test. The fungal concentration in aqueous solution before and after the contact with an oil phase (hexadecane) was found to be unchanged, indicating that fungi prefer partitioning in aqueous phase.

### DETAILED DESCRIPTION OF ILLUSTRATIVE EMBODIMENTS

This disclosure describes methods of bio-enhanced remediation using a filamentous microbe and model systems and methods for visually evaluating bio-enhanced remediation activity of a filamentous microbe in a controlled setting. The system generally uses microfluidics as a model for regions of low permeability to visually evaluate active removal of non-aqueous phase liquid (NAPL).

Non-aqueous phase liquid (NAPL) trapped in stagnant or low permeability regions, such as dead-end fractures or rock matrices, are hard to remediate because they are mostly inaccessible by groundwater flow. Existing bio-enhanced remediation technologies include using the chemotactic motility of bacteria. However, bacteria cannot cross oil-water boundaries. Hence, such methods rely on the diffusion and dissolution of contaminants from NAPL to the aqueous phase. This process is slow and limited by the interfacial area.

In contrast, this disclosure describes bio-enhanced remediation methods that exploit properties of filamentous microbes—e.g., hyphal fungi and/or filamentous bacteria. For example, hyphae of fungi can generate a tremendous turgor pressure on their tips and produce surfactants that allow the hyphae to navigate through small pores, air pockets, and even rock matrices in porous media. To date, however, there has been no direct visualization of fungal hyphae penetrating into oil-water interfaces. Thus, the utility of using fungi for bio-enhanced remediation of NAPL has been unclear.

This disclosure describes the active removal of NAPL by fungi over 40 hours. Clogging of the preferential flow path by fungi induced flow instability, which led to the viscous fingering-like displacement of trapped NAPL. Moreover, fungal hyphae effectively penetrated water-oil interfaces and significantly enhanced the oil removal from low porosity regions via direct consumption and oil displacement induced by hydrophilic hyphae-aided push. The results of this study demonstrate the use of branching fungi for bio-enhanced remediation of trapped NAPLs and, additionally or alternatively, in multi-phase flow systems containing microbes (e.g., microbial-enhanced oil recovery). Further, the study establishes a microfluidics system that can be used as a visual and quantitative assessment kit for bio-enhanced remediation.

Naphthalene-degrading fungal colonies were isolated from a local coal-tar contaminated site. Through microbiome analysis, a fungal colony was isolated that reflected the major fungal population in biofilms sampled from the site. Fungi from the colony were suspended in a minimal salt medium and injected into a “fracture-on-a-chip” microfluidic chip system that was initially saturated with a model NAPL. The microfluidic chip is designed to include fractures and surrounding low-porosity matrix regions that mimic a real fractured aquifer environment. The results showed active removal of NAPL by the fungi through various mechanisms. First, the fungus colonized the main fracture (or preferential flow path), clogging the main fracture and inducing pressure build-up upstream of the clogging, which caused the water phase to invade the oil-saturated region and displace NAPLs from the microfluidic system. Further, fungal hyphae effectively penetrated water-oil interfaces and significantly enhanced oil removal from low-porosity regions.

While described herein in the context of an exemplary embodiment in which the filamentous microbe is a hyphal fungus, the methods, compositions, and models described herein can involve the use of hyphal fungi, filamentous bacteria, or any combination of two or more filamentous microbial species.

### NAPL Displacement by Fungi

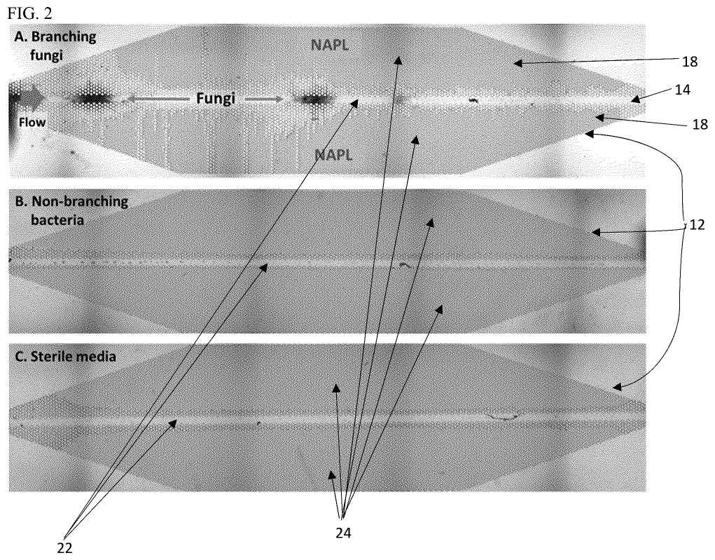

The microfluidic system designed for this study allowed observation of remarkable removal of NAPL by the fungal strain. Within 65 hours of injection, most NAPL is removed from the microfluidic bed as the fungi grow in the main flow channel and expand their hyphae into the rock matrix regions (FIG. 2A). In contrast, cases in which non-branching, naphthalene-degrading bacteria (FIG. 2B) and sterile M10 medium (FIG. 2C) are injected show insignificant oil displacement from the microfluidic bed. Later analysis indicated that this phenomenon could be attributed to two mechanisms. First, the fungal colonization of the main flow channel increases pressure upstream above the capillary pressure, thereby causing the injected water phase in the main channel to invade the trapped oil phase in the microfluidic bed. This invasion pushes the oils out of the microfluidic bed, contributing to the reduction of oil in the microfluidic system. Secondly, hyphae of fungi penetrated into the oil, which further enhanced removal due to either direct consumption of the NAPL phase by the fungus or interface push by hydrophilic hyphae.

### Oil Displacement by Channel Clogging and Pressure Build-Up

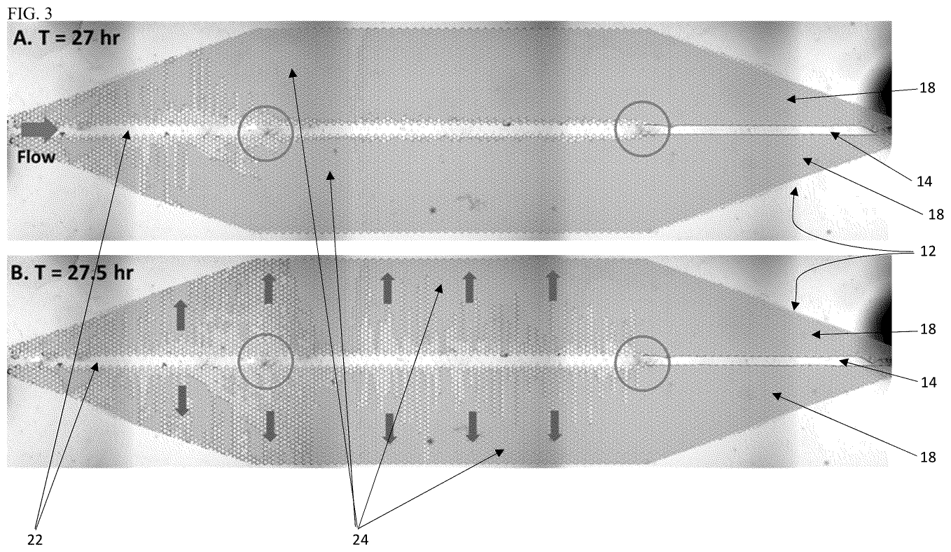

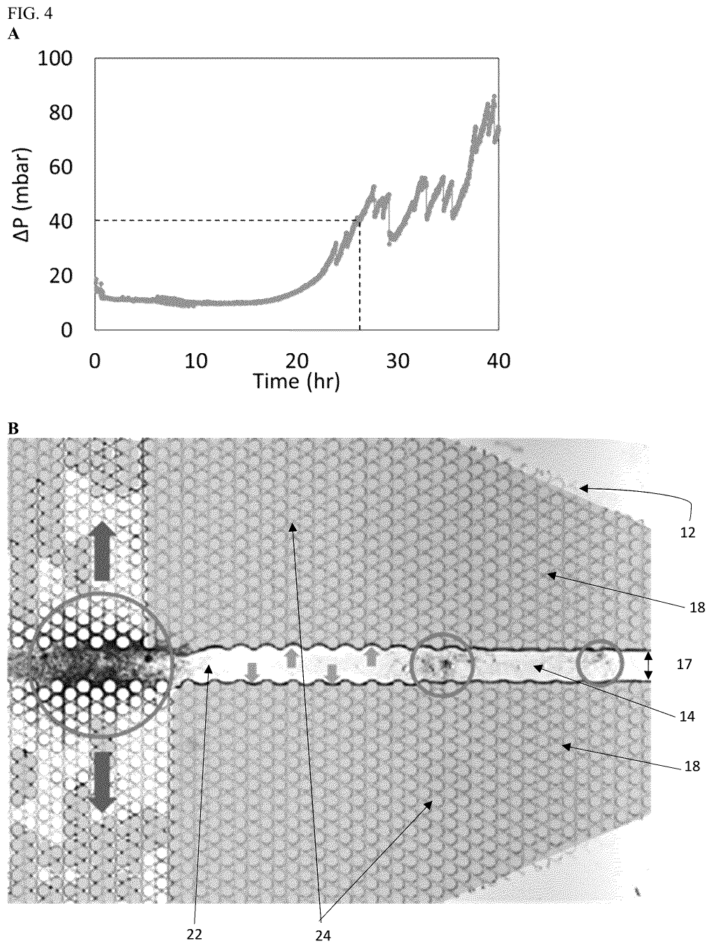

To assess the effects of pressure build-up caused by the fungal colonization, pressure sensors were installed at the inlet and outlet of the microfluidic system to allow monitoring of the pressure difference. In addition, the capillary pressure required for the water phase to invade a pore filled with oil, approximately 40 mbar, was calculated using the Young-Laplace equation. The flow experiment was performed as described in the EXAMPLES section, below. The result shows that the fungal colony clogs the main channel as it grows. Between 27 hours and 27.5 hours, the water phase upstream of the colony invades into the oil-saturated low porosity regions of the microfluidic bed (FIG. 3A and FIG. 3B). The pressure reading across the chip measured to be around 40 mbar (FIG. 4A), which corresponds to the estimated capillary pressure. Such clogging-induced pressure build-up followed by invasion can be visualized more clearly in a close-up image of the clogged area (FIG. 4B) as the extent of invasion differs by the degree of clogging.

Generalization by Conducting Experiments with Randomized Porous Media

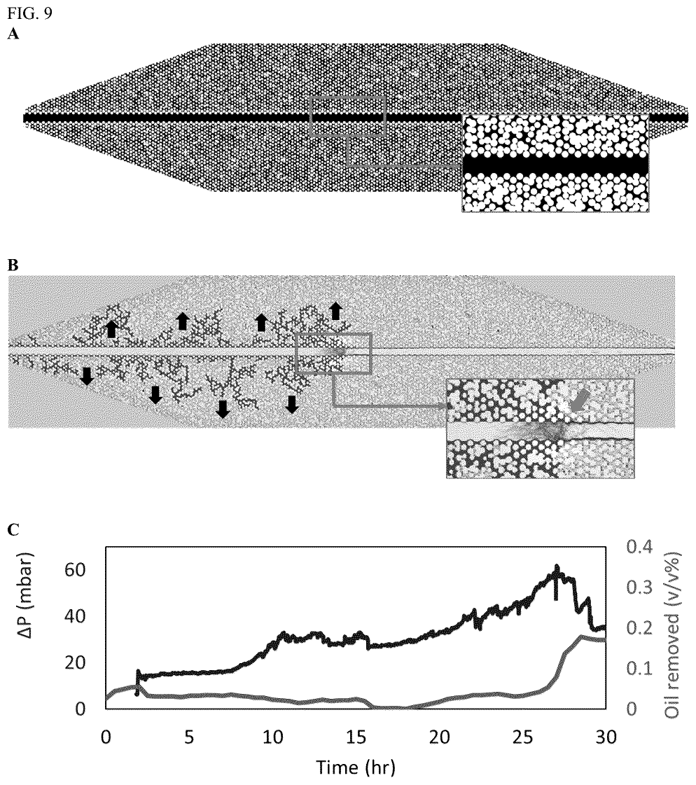

To investigate whether the observed fungi-induced pore invasion and oil displacement can be reproduced in a more realistic environment, tests were conducted in a porous media chip with randomly arranged pillars, a configuration more representative of actual porous media structures than regularly arranged pillars (FIG. 9A). The randomization of pillars is performed as follows. A random number is sampled from a normal distribution with a mean equal to either the original pillar diameter or zero, and standard deviation of either 5 μm or half the pore throat for pillar size and displacement, respectively. For randomizing the displacement, sampled numbers are added to the original x and y coordinates of the pillars in the regularly arranged chip to obtain a randomized coordinate. Randomized pillar diameter is also assigned to the pillars. The distance between the pillars (pore throat) that are interfacing the main channel is kept the same to maintain the initial pore invasion pressure the same as the regular porous media case. Results from the experiment conducted under the same condition reproduced the rapid displacement of oil from the chip as fungi colonizes the main flow channel within 30 hours of the experiment (FIG. 9B). The displaced areas are indicated by blue color. The pressure increase corresponding to the growth of the fungi was confirmed and as the pressure drop exceeded the estimated capillary pressure (40 mbar), rapid pore invasion event occurred and removed oils from the chip (FIG. 9C). The invasion region located upstream of the choke point coincides with the data in FIG. 3 with regularly arranged pillars, which confirms that the pore invasion was driven by the pressure build-up due to clogging. These results clearly show that fungi can actively divert flow and displace oils out of low-porosity regions in realistic porous media systems.

### Hyphae-Enhanced Oil Removal

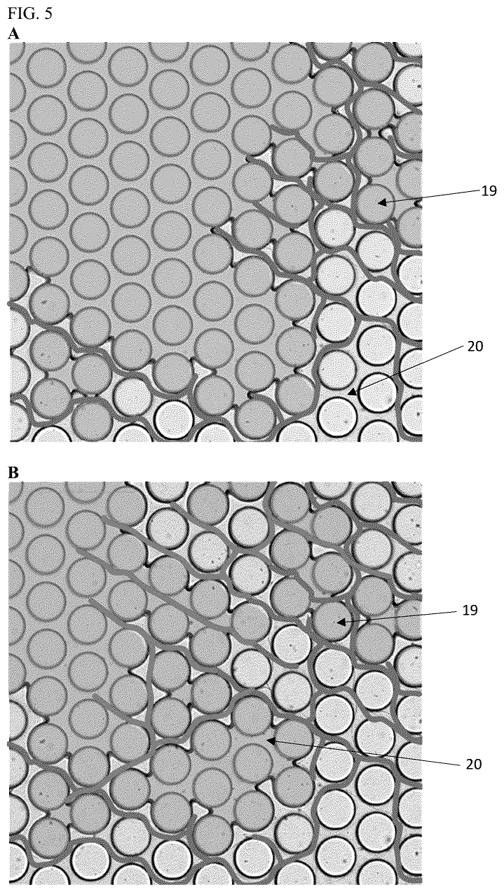

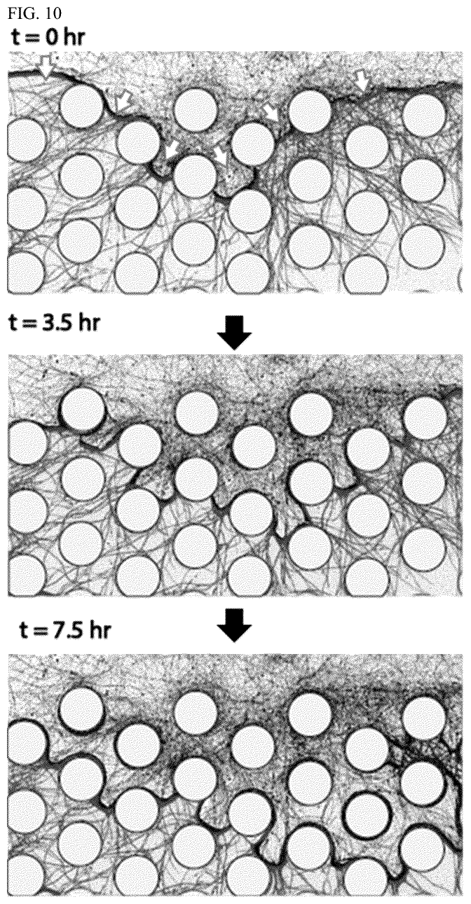

FIG. 5 shows that oil removal from the microfluidic bed is accelerated where the oil-water interface is penetrated by fungal hyphae. As fungi grow and extend their hyphae into oil in low-porosity regions, oils are removed shortly after the oil-water interface has been penetrated. While a single filament of hypha was enough to displace oil and induce water invasion at small pore throat size of 10 μm (FIG. 5), multiple fibers induce pore invasion in the case of large pore throats (100 μm; FIG. 10). This could be attributed to direct oil consumption by the fungi and/or the push of the oil-water interface by hydrophilic hyphae.

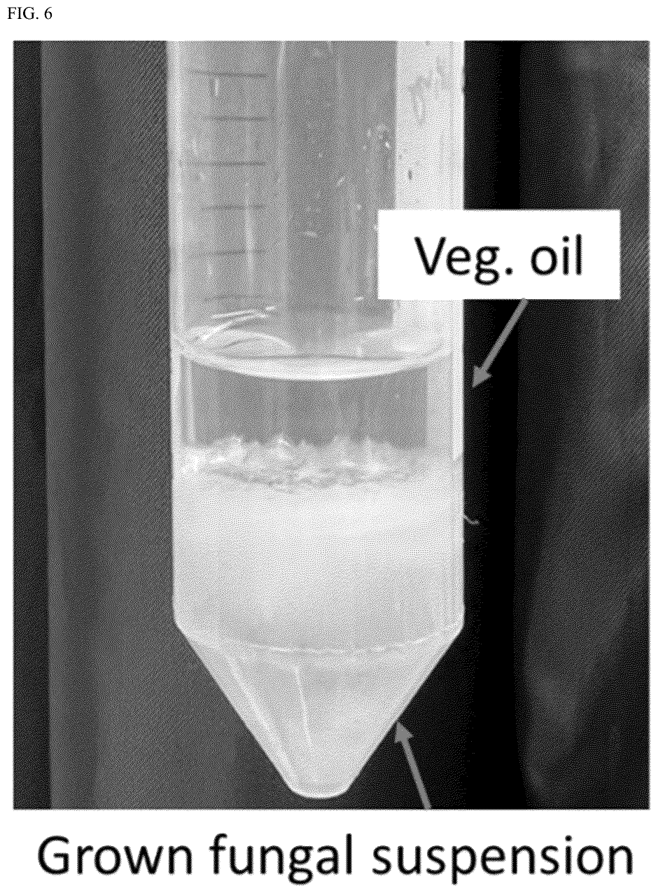

To test whether the fungi use vegetable oil as the carbon source (i.e., consume oil), a batch experiment was performed in which a fungal colony is inoculated into 20 mL of M10 medium (carbon-lacking) with 5 mL of vegetable oil. FIG. 6 shows the active growth of fungi in a condition in which vegetable oil is provided as the sole carbon source. This result confirms that the removal of oil from the microfluidic bed may be enhanced by hyphae directly consuming oil.

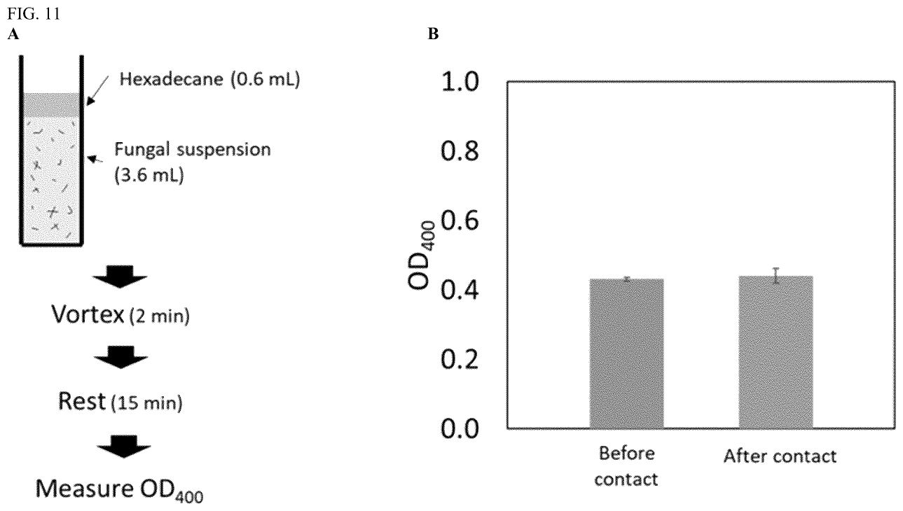

In addition, the fungal hyphae were determined to be hydrophilic using a Microbial Adsorption To Hydrocarbon (MATH) test and visual observation of the contact angle. The methodology of MATH is shown in FIG. 11A. Briefly, fungal suspension in aqueous phase is contacted with hydrocarbon phase (e.g., hexadecane). Partitioning of fungal particle to either phase is measured via analyzing optical density at 400 nm (OD₄₀₀) in the aqueous solution. OD₄₀₀ is a proxy to fungi concentration in the solution and triplicate samples were analyzed. The result shows that fungal concentration before and after contact with the oil phase remained unchanged (FIG. 11B). This indicates that fungi prefer to stay in aqueous phase which demonstrates that the fungi are hydrophilic.

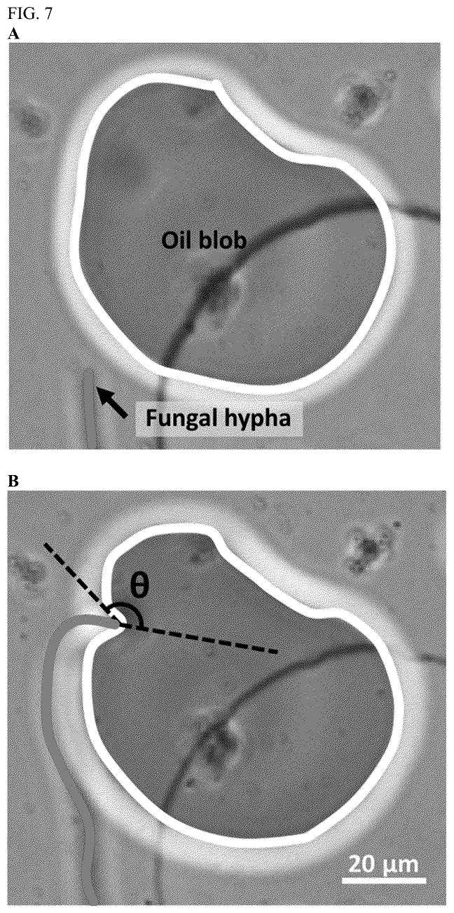

In addition, FIG. 7 provides visual confirmation of the hydrophilic nature of fungal surfaces. As the hypha approaches and pokes into an oil blob, the oil-water interface turns into a crevice-like shape (FIG. 7B). The contact angle, θ, between oil and hypha in the water phase is higher than 90°, indicating that oil is non-wetting to hypha and that hypha is hydrophilic. When a hydrophilic hypha penetrates an oil-water interface, the oil will repel away from the hypha while water will be drawn into the hypha. Consequently, the penetration pushes the oil, causing the oil-water interface to recede and displacing oil from pore spaces.

### Fungi-Enhanced Oil Remediation

This study visually demonstrates, for the first time, the role of branching fungi in remediating NAPLs trapped in low-porosity regions in subsurface environments through microfluidics. The experimental results reported herein suggest that the fungus can act in two ways: first, injected fungi can colonize the main flow channel (e.g., fracture) and build up pressures upstream above the capillary pressure causing the invasion of water phase into pores and subsequent displacement of oil out of the pore (FIGS. 3A, 3B, 4B, 8A, and 9B). Second, fungal colonies grow and extend their hyphae into the oil phase facilitating direct consumption and hydrophilic hyphae-enhanced oil displacement (FIGS. 5A, 5B, 7B, 8B, and 10). In the presence of a strong pressure gradient in the water phase (e.g., near the injection location), the former effect may be dominant as the oil-water interface will recede faster than the hyphae approaching the oil. On the other hand, in the regions further away from the injection location, the second mechanism may be more important.

These new findings provide insights into how branching fungi can facilitate the bio-enhanced remediation of trapped NAPLs. The oil displacement due to clogging and pressure build-up and hyphae-enhanced push can mobilize trapped NAPLs and make them accessible to groundwater flow and other microbes. Therefore, the hydrocarbon-degrading fungi in this study can not only seek and degrade trapped NAPLs by themselves but also work synergistically with other bioremediating microbes to accelerate the biodegradation rate of trapped NAPLs. In the case of the pump-and-treat approach, the oil displacement induced by branching fungi can improve the extraction efficiency of trapped NAPL.

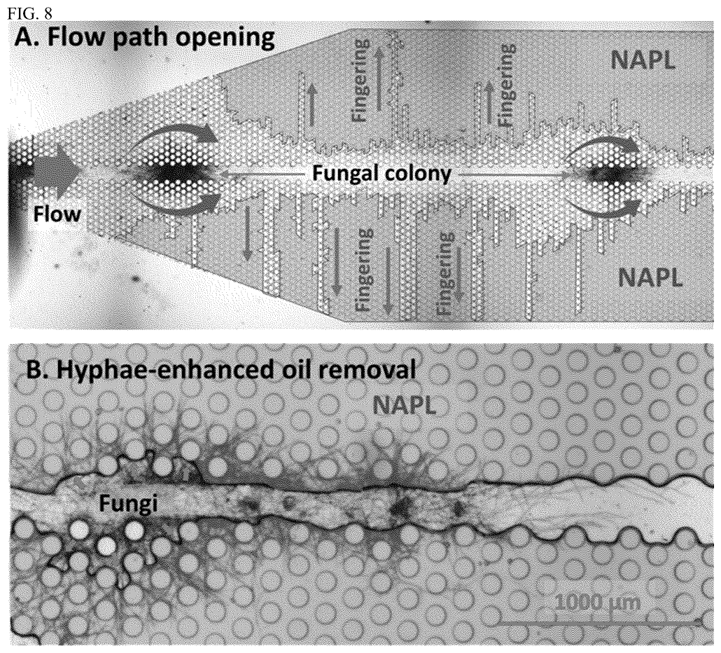

Furthermore, the microbe-induced fingering pattern of water invasion (FIG. 8A) has never been reported. Since the pattern of invasion has been an active topic of research in the oil industries, CO₂ sequestration, aquifer recharge, etc., this study provides proof-of-concept for using microbial activity in diverse multi-phase flow systems in porous media.

### Microbial-Enhanced Oil Recovery

Oil industries can exploit branching fungi-induced oil displacement in enhancing oil recovery from aquifers. Conventional microbial-enhanced oil recovery (MEOR) relies on surfactants produced by microbes that facilitate the dissolution of oil into an aqueous phase to enhance oil recovery, which requires the separation of surfactants from the pumped oil-water mixture in the later stage. However, the oil displacement by the fungal strain isolated in this study is found to proceed by purely mechanical means since no surfactant production was detected. This strategy could provide a more cost-effective and environmentally friendly approach to MEOR.

### Bio-Enhanced Remediation and Oil Removal Model System

The specially designed microfluidic system described herein allows one to directly visualize bio-enhanced remediation of contaminants and/or displacement of NAPLs by microbes in porous media flow systems. This approach can be widely used to assess how a given microbe or microbial cocktail can effectively remediate and/or displace NAPLs from a particular contaminated site. For example, the microfluidic system's flow channels, contaminant composition, and contaminant distribution can be designed to mimic the subsurface environment of a contaminated site of interest. An environmental engineer can estimate bio-enhanced remediation and/or NAPL displacement by analyzing the images and effluent and by varying parameters (e.g., microbial cocktail, flow rate), and one can identify a condition that results in maximum bio-enhanced remediation and/or NAPL displacement efficiency. Such an apparatus and process can be developed into a kit for testing bio-enhanced remediation strategies under realistic flow conditions in a cost effective way. In addition, such a kit can be implemented as a teaching tool for K-12 schools to demonstrate various subsurface flow phenomena, often delivered only conceptually due to the opaque and complex nature of the subsurface system.

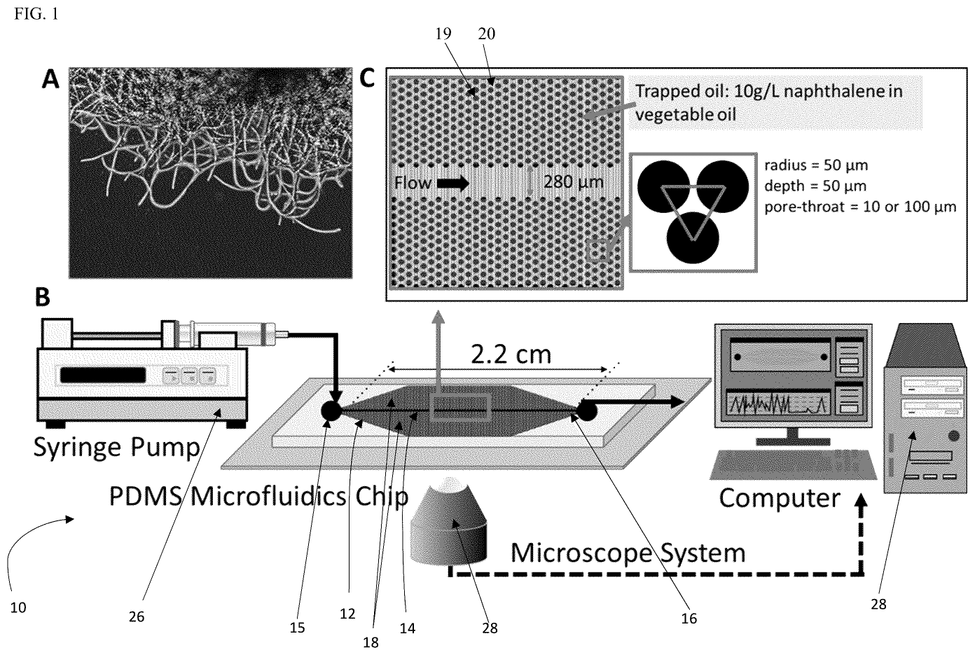

Referring to FIG. 1B, the bio-enhanced remediation model system (10) generally includes a microfluidic device (12). The microfluidic device (12) includes a main flow channel (14) that has an inlet (15), an outlet (16), and a main flow channel width (17). One or more microfluidic beds (18) are in fluid communication with the main flow channel (14). The microfluidic bed (18) includes a plurality of structures (19) that define secondary channels (20). An aqueous phase (22) is dispersed within at least a portion of the main flow channel (14). A non-aqueous phase liquid (NAPL, 24) is dispersed within at least a portion of the secondary channels (20) of the microfluidic bed (18). A fluid source (26) is in fluid communication with the inlet (15). Finally, an imager (28) is in visual communication with at least a portion of the microfluidic device (12). In one or more embodiments, the imager (28) can be in electronic communication with a computer (30).

The exemplary model system illustrated in FIG. 1B includes two microfluidic beds (18), one on either side of the main flow channel (14). The bio-enhanced remediation model system can, however, includes any number (e.g., one, two, three, four, etc.) of microfluidic beds (18), the number of which may be influenced by the shape of the main flow channel (14). While illustrated in FIG. 1B as an essentially straight linear channel, the main flow channel (14) can be designed to possess any desired shape or pathway to model a particular environmental location.

The fluid source (26) can be any suitable source of fluid that can control the flow of fluid through the inlet (15) into the main flow channel (14). Exemplary fluid sources include any container or device that is capable of holding a fluid sample and capable of being in fluid communication with the microfluidic device. Thus, exemplary fluid sources (26) include, but are not limited to, a syringe, a vial, a beaker, a jar, an ampule, a test tube, a microfuge tube, etc. The fluid source (26) can include, or be connected to, a pump or other device that can control the flow of fluid through the inlet (15) into the main flow channel (14) in an automated manner. Thus, fluid may be introduced into the system manually, automatically, or a combination of both manual and automatic control.

The main flow channel (14) has a main flow channel width (17) that can be designed to model any aspect of an environmental remediation site. The main flow channel (14) typically has a width (17) of from 5 μm to tens of centimeters.

Thus, in one or more embodiments, the main flow channel (14) can have a minimum width of at least 1 μm, at least 2 μm, at least 3 μm, at least 4 μm, at least 5 μm, at least 10 μm, at least 20 μm, at least 30 μm, at least 40 μm, at least 50 μm, at least 60 μm, at least 70 μm, at least 80 μm, at least 90 μm, at least 100 μm, at least 200 μm, at least 300 μm, at least 400 μm, at least 500 μm, at least 600 μm, at least 700 μm, at least 800 μm, at least 900 μm, at least 1 mm, at least 2 mm, at least 3 mm, at least 4 mm, at least 5 mm, at least 6 mm, at least 7 mm, at least 8 mm, at least 9 mm, or at least 10 mm.

In one or more embodiments, the main flow channel (14) can have a maximum width of no greater than 100 cm, no greater than 10 cm, no greater than 1 cm, no greater than 500 mm, no greater than 100 mm, no greater than 50 mm, no greater than 10 mm, no greater than 5 mm, no greater than 1 mm, no greater than 900 μm, no greater than 800 μm, no greater than 700 μm, no greater than 600 μm, no greater than 500 μm, no greater than 400 μm, no greater than 300 μm, no greater than 200 μm, or no greater than 100 μm.

In one or more embodiments, the main flow channel (14) can have a width, or varying widths along its length, that fall with a range having endpoints defined by any minimum width identified above and any maximum width identified above that is greater than the selected minimum width. Thus, for example the main flow channel (14) can have a width, or varying widths along the length of the channel, of from 5 μm to 10 mm, from 1 μm to 5 mm, from 1 μm to 10 cm, from 5 μm to 5 mm, etc.

In one or more embodiments, the main flow channel (14) can have a width that equals any minimum width or any maximum width identified above. Thus, for example, the main flow channel can have a width of 5 μm, 10 μm, 20 μm, 30 μm, 50 μm, 100 μm, 500 μm, 1 mm, 2 mm, 3 mm, 4 mm, 5 mm, or 10 mm.

The microfluidic bed (18) includes structures (19) that define secondary channels (20). The structure can be of any suitable size and shape. Moreover, the size and shape of any structure (19) can be independent of the size and shape of any other structure (or population of structures) in the microfluidic bed (18). Thus, while FIG. 1C illustrates an exemplary embodiment in which the structures (19) are a regular array of circular structures having a radius of 50 μm, the structures (19) in the microfluidic bed (18) need not be arranged in a regular pattern, be circular, be of the same shape, or be of the same size. Similarly, the secondary channels (20) defined by the shape and positioning of the structures (19) in the microfluidic bed (18) need not be of uniform shape, size, or pattern. The microfluidic bed (18) models regions of low permeability of an environmental remediation site. Thus, the structures (19) and secondary channels (20) of the microfluidic bed (18) can possess any design and/or arrangement appropriate to model a theoretical or actual remediation site.

In one or more embodiments, at least a portion of the secondary channels (20) have a maximum width of no greater than 1 mm, no greater than 900 μm, no greater than 800 μm, no greater than 700 μm, no greater than 600 μm, no greater than 500 μm, no greater than 400 μm, no greater than 300 μm, no greater than 200 μm, no greater than 100 μm, no greater than 90 μm, no greater than 80 μm, no greater than 70 μm, no greater than 60 μm, no greater than 50 μm, no greater than 40 μm, no greater than 30 μm, no greater than 20 μm, no greater than 10 μm, no greater than 9 μm, no greater than 8 μm, no greater than 7 μm, no greater than 6 μm, no greater than 5 μm, no greater than 4 μm, no greater than 3 μm, no greater than 2 μm, or no greater than 1 μm.

In use, the bio-enhanced remediation model system (10) further includes a microbial composition that includes at least one filamentous microbe such as a hyphal fungus. As used herein, a filamentous microbe refers to any microbe—fungal or bacterial—that grows as segmented filaments or grows hyphae. Thus, the filamentous microbe can be, but is not limited to, a fungus or a member of the phylum Actinobacteria (or Actinomycetota). The filamentous microbe can be a wild-type strain or may be genetically modified. A genetically modified filamentous microbe may be engineered to include any non-native characteristic of the wild-type strain from which the genetically modified strain is derived. Exemplary genetic modifications can include gain of function modifications that promote growth of hyphae; consumption of a remediation contaminant, a breakdown product of a remediation contaminant, or a metabolic byproduct of a remediation contaminant; or metabolism of a remediation contaminant, a breakdown product of a remediation contaminant, or a metabolic byproduct of a remediation contaminant. Exemplary genetic modifications also can include loss of function modifications that reduce or inhibit processes that would otherwise impede the microbe's ability to contribute to bio-enhanced remediation.

In one or more embodiments, the microbial composition can be a bio-enhanced remediation composition or a bio-enhanced oil recovery composition. In either aspect, the microbial composition can include a cocktail of two or more microbes, at least one of which is a filamentous microbe. Thus, in one or more embodiments, bio-enhanced remediation and/or bio-enhanced oil recovery described above may be accomplished using a single species of filamentous microbe. In one or more alternative embodiments, bio-enhanced remediation and/or bio-enhanced oil recovery can be accomplished using more than one species of microbe. In a cocktail, it is not a requirement that all microbes be filamentous microbes. Thus, bio-enhanced remediation and/or bio-enhanced oil recovery can be accomplished by a combination of the filamentous microbe colonizing aqueous flow channels in a bio-enhanced remediation site, degradation and/or displacement of the NAPL by the filamentous microbe, and degradation by another bio-enhanced remediation microbe in the microbial composition.

The bio-enhanced remediation model system can be employed to identify a filamentous microbe, or a microbial composition that includes a filamentous microbe, as a bio-enhanced remediation microbe or composition. Generally, the method includes introducing the candidate filamentous microbe into the aqueous phase of the bio-enhanced remediation model system, either alone or in a microbial composition that includes at least one additional species of microbe. One can then incubate the bio-enhanced remediation model system under conditions effective to allow members of microbial composition to colonize the main flow channel. The filamentous microbe or microbial composition can be identified as useful for bio-enhanced remediation if one observes hyphae infiltrating the secondary channels of at least one microfluidic bed.

The conditions selected for incubating the bio-enhanced remediation model system may be selected as appropriate considering, for example, the species being introduced into the system and/or the environment being modeled. Thus, in one or more embodiments, typical incubation conditions can include incubation in a medium containing a carbon source (e.g., an NAPL contaminant), one or more nutrients (e.g., nitrogen, phosphorus, etc.), one or more salts, and one or more metals. However, incubation conditions may be adjusted to accommodate the growth requirements of the microbe or microbes being tested. Additionally or alternatively, the incubation conditions may be adjusted in consideration of the carbon source(s), nutrients, salts, and/or metals present at an environmental remediation site or oil recovery site being modeled. As used herein, “environmental remediation site” refers to a non-laboratory site in need of remediation due to chemical contamination. As used herein, “oil recovery site” refers to a non-laboratory site from which a non-aqueous phase liquid (NAPL), whether considered a contaminant or a desirable recovery product, is recovered.

In one or more embodiments, the bio-enhanced remediation system can be incubated at room temperature. In one or more alternative embodiments, the bio-enhanced remediation system can be incubated at a temperature that approximates (e.g., ±5° C.) the natural environmental temperature of a selected bio-enhanced remediation site. Since remediation sites are often underground, the “natural environmental temperature” of a selected remediation site refers to the temperature at an underground location of remediation rather than a surface air temperature.

In one or more embodiments, the bio-enhanced remediation model system can include a non-aqueous phase liquid (NAPL) that is present in an environmental remediation site. Including the NAPL from an environmental remediation site allows one to determine in a controlled laboratory setting a filamentous microbe, or a combination of microbes that includes a filamentous microbe, that will promote bio-enhanced remediation at the environmental remediation site and/or promote oil recovery from an oil recovery site.

In one or more embodiments, one can perform the method just described in parallel using a plurality of bio-enhanced remediation model systems to compare a plurality of filamentous microbes or microbial compositions. For example, one can introduce a first filamentous microbe or first microbial composition into a first bio-enhanced remediation system, introduce a second filamentous microbe or second microbial composition into a second bio-enhanced remediation system, measure the degree to which filamentous microbes infiltrate the microfluidic beds of each bio-enhanced remediation model system, and select for bio-enhanced remediation of the NAPL the microbial composition with greater filamentous infiltration into the NAPL.

In another aspect, this disclosure describes a method of bio-enhanced remediation. Generally, the method includes introducing into a remediation site a bio-enhanced remediation composition that includes a filamentous microbe. In one or more embodiments, the bio-enhanced remediation composition can include a plurality of microbes, at least one of which is a filamentous microbe. In one or more embodiments, at least one microbe introduced into the remediation site may be genetically engineered. A genetically engineered microbe may be engineered to, for example, increase consumption of the NAPL in the remediation site, limit the ability of the microbe to metabolize carbon sources other than the NAPL in the remediation site, increase the microbe's fitness under environmental conditions found at the remediation site, integrating a pollution receptor into a fluorescent protein secretion circuit or bioluminescence circuit, etc.

In another aspect, this disclosure describes a method of bio-enhanced oil recovery. Generally, the method includes introducing into an oil recovery site a bio-enhanced oil recovery composition that includes a filamentous microbe. In one or more embodiments, the bio-enhanced oil recovery composition can include a plurality of microbes, at least one of which is a filamentous microbe.

In the preceding description and following claims, the term “and/or” means one or all of the listed elements or a combination of any two or more of the listed elements; the terms “comprises,” “comprising,” and variations thereof are to be construed as open ended i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be performed in any feasible order. And, as appropriate, any combination of two or more steps may be performed simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

### EXAMPLES

The present invention is illustrated by the following examples. It is to be understood that the particular examples, materials, amounts, and procedures are to be interpreted broadly in accordance with the scope and spirit of the invention as set forth herein.

### Isolation of Hydrocarbon-Degrading Fungus

The fungal strain (FIG. 1A) used in this study was sampled from biofilm formed in the granulated activated carbon bed at the treatment facility of a local coal-tar contaminated site (Minnesota Library Access Center, MLAC) located on the University of Minnesota Twin Cities campus. The fungus was isolated via a culture enrichment process described as follows. First, the sampled biomass (1 g) was inoculated in a carbon-deprived minimal salt medium (M10: 0.1 MgSO₄*7H₂O; 0.5 NH₄Cl; 0.7 Na₂HPO₄*2H₂O; 0.3 KH₂PO₄; 0.01 CaCl₂)*6H₂O; 0.005 g FeSO₄*7H₂O, 0.44 ZnSO₄*7H₂O; 0.20 CuSO₄*5H₂O; 0.17 MnSO4*2H₂O; 0.06 Na₂MoO₄*2H₂O; 0.10 H₃BO₃; and 0.08 CoCl₂*6H₂O) with a 1 mL spike of 1 mg/L of naphthalene (Sigma-Aldrich, St. Louis, MO) dissolved in ethanol (Sigma-Aldrich, St. Louis, MO) for two weeks at room temperature (25° C.). The medium spiked with naphthalene is denoted as M10-N. Subsequently, 1 mL of the culture was inoculated in a fresh M10-N medium for another two weeks. 10 μL of the final culture was spread onto M10 media agar and incubated flipped with a solid naphthalene grain placed on the lid until colonies appeared. A single colony was then retrieved and cultured in the M10-N and made into frozen stocks which were later inoculated in the M10-N medium for use in the microfluidics experiments.

### Microfluidic Chip and Experiments

Fractured aquifer environment-mimicking poly-dimethylsiloxane (PDMS) (Sylgard 184, Dow Corning Corp., Midland, MI) microfluidic chips were prepared from a SU-8 patterned silicon wafer mold fabricated via the photolithography process. The flow chip includes a straight channel surrounded by low porosity regions representing fracture and rock matrices, respectively. The matrix region is filled with equally spaced pillars having a radius of 50 μm. The space between posts or pore throat is set to either 10 μm or 100 μm, and the depth of the flow chip is 50 μm. The flow channel between the porous regions has a width of 280 μm and a length of 2.2 cm (FIG. 1C). The chip was initially saturated with 1 g/L naphthalene dissolved in vegetable oil. The fungal suspension solution was injected into the chip using a pulsation-free syringe pump (NEMESYS 290N, Cetoni GmbH, KorbuBen, Germany) at the constant flow rate of 0.7 μL/min. The interaction between NAPL and fungi was monitored through a scientific CMOS camera (Orca-Flash 4.0, Hamamatsu, Shizuoka, Japan) connected to a fully motorized epifluorescence inverted microscope system (T12-E Nikon, Tokyo, Japan) (FIG. 1). Pressure across the chip was measured by installing the pressure sensors (Elveflow, Paris, France) at the inlet and outlet of the chip.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A bio-enhanced remediation model system comprising:
a microfluidic device comprising:
a main flow channel comprising an inlet, an outlet, and a main flow channel width;
one or more microfluidic beds in fluid communication with the main flow channel, at least one microfluidic bed comprising a plurality of structures that define secondary channels, at least a portion of the secondary channels having a width narrower than the main flow channel width;
an aqueous phase dispersed within at least a portion of the main flow channel; and
a non-aqueous phase liquid (NAPL) dispersed within at least a portion of the secondary channels;
a fluid source in fluid communication with the inlet; and
an imager in visual communication with at least a portion of the microfluidic device.

2. The bio-enhanced remediation model system of claim 1, further comprising a computer in electronic communication with the imager.

3. The bio-enhanced remediation model system of claim 1, wherein the microfluidic device comprises two microfluidic beds in fluid communication with the main flow channel.

4. The bio-enhanced remediation model system of claim 1, wherein the fluid source comprises a syringe.

5. The bio-enhanced remediation model system of claim 4, wherein the syringe is controlled by a pump.

6. The bio-enhanced remediation model system of claim 1, wherein the main flow channel has a width of from 1 μm to 10 cm.

7. The bio-enhanced remediation model system of claim 1, further comprising a filamentous microbe.

8. The bio-enhanced remediation model system of claim 7, wherein the filamentous microbe is a hyphal fungus.

9. The bio-enhanced remediation model system of claim 1, further comprising a mixture of microbes, at least one of which is a filamentous microbe.

10. A method for identifying a filamentous microbe as a bio-enhanced remediation microbe, the method comprising:
providing the bio-enhanced remediation model system of claim 1;
introducing a microbial composition into the aqueous phase, the microbial composition comprising at least one filamentous microbe;
incubating the bio-enhanced remediation model system under conditions effective to allow members of microbial composition to colonize the main flow channel;
observing hyphae in the secondary channels of at least one microfluidic bed; and
identifying the filamentous microbe as a bio-enhanced remediation microbe.

11. The method of claim 10, wherein the NAPL comprises a compound present at a bio-enhanced remediation site.

12. The method of claim 10, wherein the microbial composition comprises a plurality of microbes, at least one of which is a filamentous microbe.

13. A method of comparing bio-enhanced remediation of a non-aqueous phase liquid (NAPL) by different bio-enhanced remediation microbial compositions, the method comprising:
providing a first bio-enhanced remediation model system of claim 1, comprising the NAPL in the microfluidic bed;
introducing a first microbial composition into the aqueous phase of the first bio-enhanced remediation model system, the first microbial composition comprising at least one filamentous microbe;
incubating the first bio-enhanced remediation model system under conditions effective to allow members of first microbial composition to colonize the main flow channel of the first bio-enhanced remediation model system;
measuring hyphae infiltration into the NAPL of at least one microfluidic bed of the first bio-enhanced remediation model system;
providing a second bio-enhanced remediation model system of claim 1, comprising the NAPL in the microfluidic bed;
introducing a second microbial composition into the aqueous phase of the second bio-enhanced remediation model system, the second microbial composition comprising at least one filamentous microbe;
incubating the second bio-enhanced remediation model system under conditions effective to allow members of second microbial composition to colonize the main flow channel of the second bio-enhanced remediation model system;
measuring hyphae infiltration into the NAPL of at least one microfluidic bed of the second bio-enhanced remediation model system; and
selecting for bio-enhanced remediation of the NAPL the microbial composition with greater hyphae infiltration into the NAPL.

14. The method of claim 13, wherein the first microbial composition or the second microbial composition comprises a plurality of different microbes, at least one of which is a filamentous microbe.

15. A method of comparing bio-enhanced remediation of a non-aqueous phase liquid (NAPL) by different bio-enhanced remediation microbial compositions, the method comprising:
providing a first bio-enhanced remediation model system of claim 1, comprising the NAPL in the microfluidic bed;
introducing a first microbial composition into the aqueous phase of the first bio-enhanced remediation model system, the first microbial composition comprising at least one filamentous microbe;
incubating the first bio-enhanced remediation model system under conditions effective to allow members of first microbial composition to colonize the main flow channel of the first bio-enhanced remediation model system;
measuring removal of NAPL from at least one microfluidic bed of the first bio-enhanced remediation model system;
providing a second bio-enhanced remediation model system of claim 1, comprising the NAPL in the microfluidic bed;
introducing a second microbial composition into the aqueous phase of the second bio-enhanced remediation model system, the second microbial composition comprising at least one filamentous microbe;
incubating the second bio-enhanced remediation model system under conditions effective to allow members of second microbial composition to colonize the main flow channel of the second bio-enhanced remediation model system;
measuring removal of NAPL from at least one microfluidic bed of the second bio-enhanced remediation model system; and
selecting for bio-enhanced remediation of the NAPL the microbial composition with greater NAPL removal.

16. The method of claim 15, wherein the first microbial composition or the second microbial composition comprises a plurality of different microbes, at least one of which is a filamentous microbe.

17. A bio-enhanced remediation composition comprising a filamentous microbe.

18. The bio-enhanced remediation composition of claim 17, wherein the filamentous microbe is genetically modified.

19. The bio-enhanced remediation composition of claim 17, further comprising a second species of microbe.

20. The bio-enhanced remediation composition of claim 19, wherein the second species of microbe is a filamentous microbe.

21. The bio-enhanced remediation composition of claim 19, wherein at least one species of microbe is genetically modified.

22. The bio-enhanced remediation composition of claim 21, wherein the genetic modification increases consumption of a non-aqueous phase liquid (NAPL), decreases ability of the microbe to metabolize carbon sources other than the NAPL, increases fitness of the microbe under selected environmental conditions, integrates a pollution receptor into a fluorescent protein secretion circuit, or integrates a pollution receptor into a bioluminescence circuit.

23. A method of bio-enhanced remediation, the method comprising introducing into a bio-enhanced remediation site the bio-enhanced remediation composition of claim 17.

24. The method of claim 23, wherein the filamentous microbe is genetically modified to metabolize at least one contaminant of the bio-enhanced remediation site.

25. The method of claim 23, wherein the composition comprises a plurality of different microbes, at least one of which is a filamentous microbe.

26. A bio-enhanced oil recovery composition comprising a filamentous microbe.

27. The bio-enhanced oil recovery composition of claim 26, further comprising a second species of microbe.

28. The bio-enhanced oil recovery composition of claim 27, wherein the second species of microbe is a filamentous microbe.

29. A method of recovering oil from an oil recovery site, the method comprising introducing into the oil recovery site the bio-enhanced oil recovery composition of claim 26.

30. The method of claim 29, wherein the composition comprises a plurality of different microbes, at least one of which is a filamentous microbe.
